(19) United States
(12) Patent Application Publication
(10) Pub. No.: US 2004/0132707 A1
(43) Pub. Date: Jul. 8, 2004

(54) Catecholate beta-lactam conjugates, method for producing the same and the use thereof

(75) Inventors: Lothar Heinisch, Jena (DE); Steffen Wittmann, Jena (DE); Ina Scherlitz-Hofmann, Falkensee (DE); Thomas Stoiber, Jena (DE); Albrecht Berg, Saalburg (DE); Ute Moellmann, Jena (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/651,251

(22) Filed: Aug. 29, 2003

Related U.S. Application Data
(63) Continuation of application No. PCT/EP02/02070, filed on Feb. 27, 2002.

(30) Foreign Application Priority Data
Mar. 1, 2001 (DE) 101 11 160.6

(51) Int. Cl. A61K 31/545; A61K 31/43

(52) U.S. Cl. 514/192; 514/200; 540/215; 540/304

(57) ABSTRACT

Catacholate beta-lactam conjugates, methods for producing these compounds, and compositions containing these compounds useful as siderophores or for treatment of bacterial infections are provided.

### CROSS REFERENCE TO RELATED APPLICATIONS

This application is a continuation of International Patent Application No. PCT/EP02/02070, filed Feb. 27, 2002, designating the United States of America, and published in German as WO 02/070016, the entire disclosure of which is incorporated herein by reference. Priority is claimed based on Federal Republic of Germany patent application no. DE 101 11 160.6, filed Mar. 1, 2001.

### BACKGROUND AND SUMMARY OF THE INVENTION

The present invention relates to new antibiotic conjugates containing catecholate derivatives derived from multibase secondary amino acids and analogous structures. The inventive compounds are effective as antibacterials, especially against Gram-negative bacteria, where the antibiotics can be transferred in the bacterial cell via iron transport routes and thereby their antibacterial activity with diminished side effects can be improved or even extended significantly more than that of known compounds of this kind. With it a contribution shall be given to combat penetration-related antibiotic resistance important in therapy of bacterial infections.

Compounds of the general formula I with the named substituents were not described in the literature so far.

It is known that some catecholate structures play an important role as iron complexing structures in natural siderophores (“Iron Transport in Microbes, Plants and Animals”, Eds.: Winkelmann, G., van Helm, D., Neilands, J. B., V.Ch.-Verlagsgesellschaft Weinheim, 1987), e.g. enterobactin, a siderophore of E. coli and other bacteral strains is a trimer of N-(2,3-dihydroxybenzoyl)-L-serine.

The monomer is also effective as siderophore (Hantke, K., FEMS Microbiol. Lett. 67 (1990), 5). The N-(2,3-dihydroxybenzoyl)glycine was found as siderophore of B. subtilis (Ito, T., Neilands, J. B., J. Amer. Chem Soc. 80 (1958), 4645).

Some catecholate substituted amino acid derivatives were prepared synthetically, e.g. N-(2,3-dihydroxybenzoyl)-L-threonine (Kanai, F.; Kaneko, T., Morishima, H., Isshiki, K., Takita, T., Takeuchi, T., Umezawa, H., J. Antibiot. 38 (1985), 39), N²,N⁶-Bis-(2,3-dihydroxybenzoyl)-L-lysine (Corbin, J. L., Bulen, W. A., Biochemistry 8 (1969), 757; McKee, J. A., Sharma, S. K., Miller, M. J.; Bioconjugate Chem., 2 (1991) 281) and N²,N⁶-Bis-(2,3-dihydroxybenzoyl)-lysyl-N⁶-(2,3-dihydroxybenzoyl)-lysine (Chimiak, A., Neilands, J. B., Structure and Bonding, 58 (1984), 89).

Some O-acylated catecholate compounds derived from mono- and diaminoacids (Heinisch L., Schnabelrauch M., Möllmann U., Reissbrodt R., DE 19654920 A1) and benzoxazine-2,4-dione derivatives derived of these catecholate compounds (Heinisch L., Wittmann S., Möllmann U., Reissbrodt R., EP 0 863 139 A1) are known. Some derivatives of mulitibase secondary amino acids derived from the last compounds are described. The designed catecholate derivatives are coupled with antibiotics to conjugates highly effective as antibacterials in vitro.

Catecholates of di- and triamino compounds, linear or tripodal, without carboxyl group are described, e.g. triscatecholate derivatives of Bis-aminopropyl-amine (Martell, A., E.; Motekaitis, R. J., Murase, I.; Sala, L. F., Stoldt, R. Ng, Chiu, Y., Rosenkrantz, H.; Inorg. Chim. Acta (1987), 138, 215-30.), Bis-catecholate derivatives of spermidine (Bergeron R. J., Burton P. S., McGovern K. A., Onge E. J. St.; J. Med. Chem. 1980, 23, 1130-1133) and myxocheline derivatives (Ambrosi H. D., Hartmann V., Pistorius D., Reissbrodt R., Trowitzsch-Kienast W.; Eur. J. Org. Chem. 1998, 541-551).

As dibasic secondary amino acids or analogous structures used as backbone of the inventive compounds only aminoethyl- and aminopropylglycine prepared from the amine and glyoxylic acid by catalytic hydrogenation are described (Byk, G., Gilon, Ch.; J. Org. Chem. 57, 5687-5692 (1992), Will D. G., Breipohl G., Langner D., Knolle J., Uhlmann E., Tetrahedron 51, 12069-12082, 8, 1995).

Of multibase secondary amino acids also used as backbone only a Co-III-complex of N′-(aminoethyl)-N-aminoethylglycine (3,7,11-tri-aza undecanoic acid) is described (Watanabe, Kuroda Nippon Kagaku Kaishi, 1972, 1409-1415, Chem. Abstr. 77, 121610x, 1972). Other multibase secondary amino acids are unknown.

Several catecholate compounds were coupled with β-lactams, whereby an important increase of the antibacterial activity of these antibiotics was achieved due to transfer of the antibiotics in the bacterial cell via iron transport routes (Arisawa, M., Sekine, Y., Shimizu, S., Takano, H., Angehm, P., Then, R. L., Antimicrob. Agents Chemother. 35 (1991), 653).

Until now such compounds were not used for human therapy, partly because of negative side effects.

To reach this aim new effective synthetic siderophores must be found which are suitable to forming highly antibacterial active conjugates with antibiotics, especially active against resistant pathogens like Stenotrophomonas maltophilia, and which exhibit less side effects as known compounds of this kind.

The underlying object of the present invention is to discover new catecholate antibiotic conjugates derived from multibase secondary amino acids or analogous structures and the use thereof. The invention is focused on the development of compounds suitable for transfer of antibiotics into the bacterial cell and which surpass the activity of known compounds of this kind.

By use of acylated catecholate compounds or by transforming of the catecholate structure into the benzoxazine structure it should be reached, that the compounds achieve improved pharmacological properties or that they can be used as pharmacological transport forms for the actual penetration promoting catecholate compounds.

The aim of the invention is to detect new antibiotic conjugates of catecholate compounds or its acylated derivatives or into benzoxazine structures transformed derivatives derived from secondary amino acids or analogous structures of the general formula I, which possess higher antibacterial activity as known compounds of this kind.

### DETAILED DESCRIPTION OF THE EMBODIMENTS

New antibiotic conjugates, especially penicillin and cephalosporin conjugates of catecholate compounds, their acylated derivatives or into benzoxazine structures transformed derivatives derived from secondary amino acids or analogous structures are provided by the general formula I

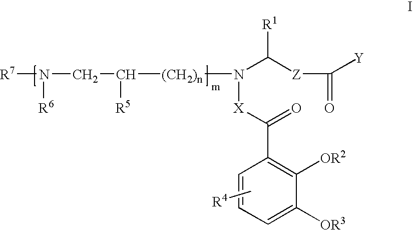

wherein

R¹=H, alkyl, substituted alkyl, aryl, substituted aryl,

R²=H, COalkyl, COOalkyl,

X=direct bond, (CH₂)_{q}NH—, CO(CH₂)_{q}NH— with q=1-6, or

R² represents together with X a group

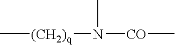

or

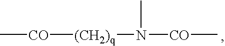

wherein q=1-6,

R³=H, COAlkyl, COOAlkyl,

R⁴=H, alkyl, substituted alkyl, aryl, substituted aryl, halogen, alkoxy, substituted alkoxy, for all possible positions, where the named substituents can be present multiple,

R⁵=H, OH, Oalkyl, Oacyl, Oaryl, alkyl, substituted alkyl, aryl, substituted aryl,

R⁶=

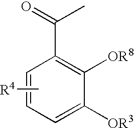

wherein R⁸=H, COalkyl, COOalkyl,

=R⁹

or R⁶=

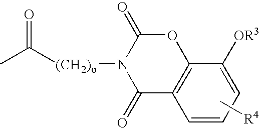

with o=1-10=R¹⁰

or R⁶=

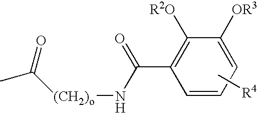

with o=1-10=R¹¹

or R⁶ and R⁷ together=

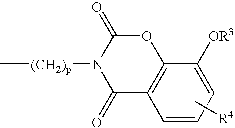

=R¹²

or R⁶ and/or R⁷=

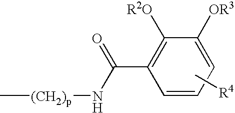

=R¹³

with p=2-10,

or R⁷=H, alkyl, substituted alkyl, aryl, substituted aryl

or R⁷=

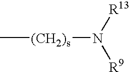

s=2-4=R¹⁴

n=0-8, m=1-3,

Y=the residue of a β-lactam antibiotic, preferably a penicillin derivative, especially a residue of ampicillin or amoxicillin (formula A) or a bacampicillin residue α-ethoxycarbonyloxyethylester of ampicillin), or a cephalosporin residue, especially a cefaclor residue (formula B),

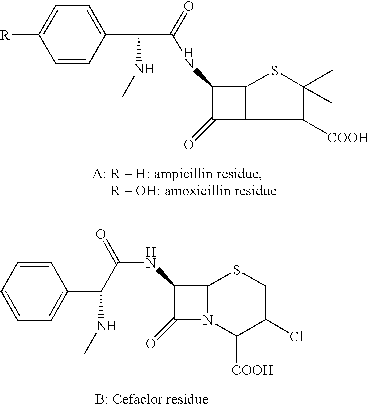

Z=direct bond, or

Z=—(CH₂)ᵣ— with r=0-10 or

Z=arylene or substituted arylene, especially

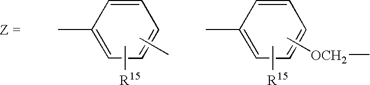

with R¹⁵=H, alkyl, substituted alkyl, aryl, substituted aryl, halogen, alkoxy, substituted alkoxy, in all possible positions.

In the above formulae and hereinafter the term acyl represents in particular C₁-C₄-alkanoyl or C₁-C₄-alkoxy-carbonyl, alkyl and alkoxy, also in complex terms as alkoxycarbonyl, in particular for C₁-C₈-alkyl or -alkoxy, alkyl substituted by halogen, alkoxy, hydroxy, carboxy and alkoxycarbonyl, alkoxy substituted by halogen, alkoxy, carboxy and alkoxycarbonyl, aryl, preferentially phenyl or phenyl substituted by alkyl, halogen, alkoxy, hydroxy, carboxy and alkoxycarbonyl, and a substituted ammoniumion, preferentially substituted single or multiple by alkyl, like single or fourfold substituted ammonium ion.

If asymmetric C atoms are present, the invention likewise relates to the corresponding D- and L-forms, enantiomers and diastereomers, and to racemates and mixtures of enantiomers and diastereomers. The compounds can represent free acids, salts or easily cleavable as well as under physiological conditions cleavable esters.

The compounds of the formula I according to the invention can be prepared by reaction of compounds of the formula I with Y=OH with a corresponding antibiotic, especially with a penicillin derivative or a cephalosporin derivative, especially with ampicillin, amoxicillin or cefaclor according to suitable procedures as by the anhydride method (e.g. by isobutyl chloroformate), or by the active ester method (e.g. by N-hydroxysuccinimide and dicyclohexyl-carbodiimide) or by the chloride method resulting in the compounds of the formula I.

Compounds of the formula I with Y=OH were prepared as follows: In a first step the secondary amino acids of the formula IV were prepared by reaction of the corresponding amine II with the α-keto acids III, wherein R represents H or (CH₂)ₚ—NH₂

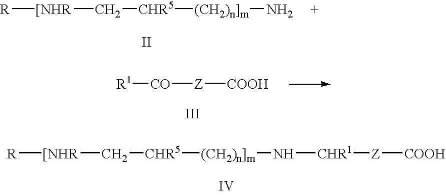

The compounds IV can be purified by preparation of the carbobenzoxy derivatives (Z-derivatives), separation of side products by HPLC and cleaving of the Z-groups hydrogenolytically (by H₂/Pd/C).

In a second step the compounds of the formula I with Y=OH were prepared by reaction of the secondary amino acids with corresponding catecholate derivatives, e.g. with dihydroxy- or diacyloxybenzoic acids or their chlorides or with corresponding spacer compounds as with 8-methoxycarbonyloxy-3,4-dihydro-2H-1,3-benzoxazin-3-yl-acetyl chloride, (R⁶ or R⁷) according to suitable methods, e.g. by the anhydride method (e.g. using isobutyl chloroformate), or by the active ester method (e.g. using N-hydroxysuccinimide and dicyclohexylcarbodiimide) or by the chloride method.

In several cases the preparation of benzyl ester of IV can be advantageously. This compound can be coupled with the catecholate component according to suitable methods to the benzyl ester of formula I (Y=OCH₂C₆H₅). Than the benzyl group must be cleaved by hydrogenation.

The compounds according to the invention of the formula I with a carboxyl group can be represented as free acids, as salts or as easily cleavable esters, especially cleavable under physiological conditions. The compounds can be further purified according to suitable methods, e.g. by crystallisation or by chromatographic methods.

The compounds according to the invention of the formula I show antibacterial activity surpassing the activity of known comparable compounds. The antibacterial activity was tested by a microdilution assay according to National Committee for Clinical Laboratory Standards, 1998, Methods for dilution antimicrobial susceptibility tests for bacteria that grow aerobically, Approved standard M7-A, NCCLS, Villanova, Pa.

The minimal inhibition concentrations (MICs) were determined for the following bacterial strains: The Gram-negative strains Pseudomonas aeruginosa SG 137, ATCC 27853, Escherichia coli ATCC 25922, Klebsiella pneumoniae ATCC 10031, Stenotrophomonas maltophilia GN 12873, Serratia marcescens SG 621 as well as for the Gram-positive strain Staphylococcus aureus SG 511.

The results of the antibacterial assays are described in the table. For comparison the respective data of azlocillin, ampicillin and meropenem were included. The results show, that the compounds according to the invention possess much higher antibacterial activity as azlocillin, in many cases also as highly efficient meropenem. Especially the excellent activity against the species Stenotrophomonas maltophilia, resistant against meropenem, is remarkable. Comparable high activities were also achieved against Burkholderia strains.

Thus, the compounds according to the invention bacterial resistance can be overcome successfully. Compounds with Z=phenylene showed a broad activity against Gram-negative and surprisingly in comparison to compounds of compounds of this group so far also against Gram-positive bacteria. In combination with a β-lactamase inhibitor activities against MRSA and mycobacteria could be demonstrated.

Some compounds according to the invention showed much higher activity against Gram-negative bacteria than known catecholate-β-lactam conjugates according to the literature given above, also against the strain Stenotrophomonas maltophilia, which is a difficult pathogen.

The compounds of the general formula I are suitable as therapeutics for bacterial infections due to their antibacterial properties. In such diseases the compounds of the formula I can be applied either on their own or in the form of pharmaceutical preparations with physiologically compatible adjuvants or carrier materials which are known in the art, wherein all customary pharmacological forms of application are possible in principle.

### EXAMPLES

### Example 1

N-[3,7-Bis-(2,3-dimethoxycarbonyloxybenzoyl)-3,7-diaza-octanoyl]-ampicillin

Formula I with R¹, R⁴, R⁵=H; R², R³=COOCH₃, R⁶=R⁹ with R⁸=COOCH₃, R⁷=CH₃, n=1, m=1, X and Z=direct bond, Y=ampicillino.

To a solution of 0.651 g (1 mmol) 3,7-bis-(2,3-dimethoxycarbonyloxybenzoyl)-3,7-diaza-octanoic acid and 0.112 ml N-methylmorpholine in 10 ml absolute tetrahydrofuran 0.131 ml (1 mmol) isobutyl chloroformate were added at −20° C. with stirring. The mixture was stirred 1 hour at −10° C. and than a solution of 0.453 g (1.1 mmol) ampicillin trihydrate and 0.153 ml (1.1 mmol) triethylamine in 4 ml tetrahydrofuran and 1 ml water were added. The mixture was stirred 1 hour at −10-0° C. and 1 hour at 20° C. and than evaporated under vacuum. The residue was dissolved in ethyl acetate and water and the solution was acidified carefully with HCl at 0-5° C. and shaken. The organic phase was separated, washed with brine to pH 7, dried and evaporated under vaccum. The residue was purified by preparative HPLC on silicagel (Eurospher 100 C18, 7 μm, Fa. Knauer, Berlin) with a mixture of acetonitrile/(water (37.5/62.5) as eluent. The acetonitrile was evaporated and the residue dried by lyophilisation to give 0.44 g (45%) of the title compound as a colourless solid.

¹H NMR (DMSO-d₆): 1.40 (s, 3H, CH₃); 1.53 (s, 3H, CH₃); 1.75 (m, 2H, CH₂); 2.76 (s, 3H, CH₃); 2.96-3.40 (m, 4H, 2×CH₂); 3.77-4.20 (m, 14H, 4×0 CH₃, 1×CH₂COOH); 4.17 (s, 1H, 3-CH); 5.38 (m, 1H, 7-CH); 5.50 (m, 1H, α-CH); 5.72 (q, 1H, 6-CH); 7.25-7.60 (m, 11H, ArH), 8.71, 9.15 (m, 1H, NHCO).

For the preparation of the sodium salt a solution of 0.02 g sodium ethylhexanoate in 3 ml ethyl acetate was added to a solution of 0.10 g of the title compound in 12 ml ethyl acetate. The precipitate was filtered off after 10 min. and washed with petroleum ether to give the sodium salt of the title compound as colourless solid, yield 90%.

### Example 2

N-[3,10,17-Tris-(2,3-diacetoxybenzoyl)-3,10,17-triaza-heptadecanoyl]-ampicillin

Formula I with R¹, R⁴, R⁵, R⁷=H; R², R³=COCH₃, R⁶=R⁹ with R⁸=COCH₃, n=4, m=2, X and Z=direct bond, Y=ampicillino.

The title compound (2) was obtained analogous to example 1 from 3,10,17-tris-(2,3-diacetoxybenzoyl)-3,10,17-triaza-heptadecanoic acid and ampicillin in a yield of 40% as a colourless solid. ¹H NMR (CDCl₃): 1.15; 1.49 (m, 22H, 8×CH₂ and 2×CH₃); 2.15-2.2912 (m, 18H, 6×COCH₃); 3.07-3.38 (m, 8H, 4×NCH₂,); 4.29 (1H, s, CH); 5.40 (1H, d, CH); 5.54 (m, 1H, CH); 6.40, 6.50 (2×d, 1H; CH); 7.14-7.30 (m, 14H, aromat.).

### Example 3

N-[6-Bis[2-(8-methoxycarbonyloxy-benzoxazin-2,4-dion-3-yl)-ethyl]-3-[2,3-di-(methoxycarbonyloxy)-benzoyl]-3,6-diaza-hexanoyl]-ampicillin

Formula I with R¹, R⁴, R⁵=H; R², R³=COOCH₃, R⁶ and R⁷=R¹² with p=2, n=0, m=1, X and Z=direct bond, Y=ampicillino.

A mixture of 2.47 g (2.75 mmol) 6-bis-[2-(8-methoxycarbonyloxy-benzoxazin-2,4-dion-3-yl)-ethyl]-3-[2,3-di-(methoxycarbonyloxy)-benzoyl]-3,6-diaza-hexanoic acid, 0.317 g (2.75 mmol) N-hydroxysuccinimide and 0.568 mg (2.75 mmol) dicyclohexylcarbodiimide in 40 ml absolute dioxan was stirred 45 min at 0° C. and 1.5 hours at 20° C. and than kept over night at 4° C. The obtained precipitate was filtered and extracted with dioxan The extract was evaporated and dried under vaccuum. The solution of the residue in 10 ml absolute tetrahydrofuran was added to a solution of 0.727 g (1.8 mmol) ampicillin trihydrate and 0.25 ml triethylamine in 40 ml aqueous tetrahydrofuran (80%) slowly at 0° C. The mixture was stirred 45 min at 0° C. and 1.5 hours at 20° C. and than evaporated under vaccuum. The residue was dissolved in ethyl acetate/water, the solution was acidified with 1 M HCl to pH 3 and shaken. The organic phase was separated, washed with brine, dried over sodium sulfate and evaporated. To the residue was added petroleum ether to give 1.5 g (45%) of the title compound as colourless solid. The compound was purified by preparative HPLC on silicagel (Eurospher 100 C18, 7 μm, Fa. Knauer, Berlin) with a mixture of acetonitrile/water (37.5/62.5) as elutant. From the corresponding fraction the acetinitrile was evaporated under vaccuum and the residue was dried by lyophilisation to give 0.5 g (15%) of the purified title compound.

¹H NMR (DMSO-d₆): 1.41; 1.55 (s, 6H, 2×CH₃); 2.51-3.12 (m, 6H, NCH₂); 3.75-3.91 (m, 18H, 3×NCH₂, 4×COOCH₃); 4.24 (s, 1H, CH); 5.37 (t, J=4.2, 1H, CH); 5.50 (m, 1H, CH); 5.75 (2×d, 1H, CH); 7.24-7.47 (m, 10H, aromat.); 7.70 (d, 2H, aromat.); 7.80 (d, 2H, aromat.), 8.69 (2×d, 1H, NHCO); 9.13 (2×d, 1H, NHCO).

The preparation of the sodium salt was performed analogous to example 1.

### Example 4

N-[3,10,17-Tris-(2,3-diacetoxybenzoyl)-3,10,17-triaza-heptadecanoyl]-cefaclor

Formula I with R¹, R⁴, R⁵, R⁷=H; R², R³=COCH₃, R⁶=R⁹ with R⁸=COCH₃, n=4, m=2, X and Z=direct bond, Y=cefacloro.

The title compound (4) and the corresponding sodium salt were obtained analogous to example 1 from 3,10,17-tris-(2,3-diacetoxybenzoyl)-3,10,17-triaza-heptadecanoic acid and cephaclor as a colourless solid, yield 40%.

¹H NMR (DMSO-d₆): 1.48-0.80 (m, 16H, 8×CH₂); 2.14-2.26 (m, 18H, 6×CH₃); 3.20 (m, 8H, CH₂N); 3.45 (dd, 2H, CH₂); 3.75 (m, 2H, CH₂N); 4.93 (m, 1H, CH); 5.48 (m, 1H, CH); 5.68 (m, 1H, CH); 7.19-7.45 (m, 14H, aromat.); 8.30 (m, 1H, NHCO); 8.66 (m, 1H, NHCO); 9.29 (m, 1H, NHCO).

### Example 5

N-[3,10,17-Tris-(2,3-diacetoxybenzoyl)-3,10,17-triaza-heptadecanoyl]-bacampicillin

Formula I with R¹, R⁴, R⁵, R⁷=H; R², R³=COCH₃, R⁶=R⁹ with R⁸=COCH₃, n=4, m=2, X and Z=direct bond, Y=bacampicillino.

The title compound (5) was obtained analogous to example 1 from 3,10,17-tris-(2,3-diacetoxybenzoyl)-3,10,17-triaza-heptadecanoic acid and bacampicillin as a colourless solid, yield 30%.

¹H NMR (DMSO-d₆): 1.19-1.52 (m, 28H, 8×CH₂, 4×CH₃); 2.15-2.49 (m, 18H, COCH₃); 2.90-3.20 (m, 8H, CH₂N); 4.12 (m, 2H, OCH₂); 4.30 (m, 1H, CH); 5.42 (t, 1H, CH); 5.50 (m, 1H, CH); 5.70 (m, 1H, CH); 6.6.67 (m, 1H, OCH); 7.16-7.41 (m, 14H, aromat.); 8.22 2×t, 1H, NHCO); 8.66 (m, 1H, NHCO), 9.18 (m, 1H, NHCO).

### Example 6

N-[3,7,11-Tris-(2,3-diacetoxybenzoyl)-3,7,11-triaza-undecanoyl]-ampicillin

Formula I with R¹, R⁴, R⁵, R⁷=H; R², R³=COCH₃, R⁶=R⁹ with R⁸=COOCH₃, n=1, m=2, X and Z=direct bond, Y=ampicillino.

The title compound (6) and the corresponding sodium salt were obtained analogous to example 1 from 3,7,11-tris-(2,3-diacetoxybenzoyl)-3,7,11-triaza-undecanoic acid and ampicillin as a colourless solid, yield 40%.

¹HNMR (DMSO-d₆): 1.40 (s, 3H, CH₃); 1.54 (s, 3H, CH₃); 1.68-1.70 (m, 4H, 2×CH₂); 2.14-2.27 (m, 18H, 6×COCH₃), 2.98-3.30 (m, 8H, 4×NCH₂), 3.80 (s, 2H, NCH₂COOH); 4.19 (s, 1H, 3-CH); 5.40 (t, J=3.7 Hz, 1H, 7-CH); 5.50 (m, 1H, α-CH); 5.52 (m, 1H, 6-CH); 7.22-7.46 (m, 14H, ArH), 8.25-8.30 (m, 1H, NHCO), 8.60-8.80 (2×q, 1H, NHCO), 9.12-9.20 (q, 1H, NHCO).

### Example 7

N-[3,7-Bis-(5-chlor-2,3-dimethoxycarbonyloxybenzoyl)-3,7-diaza-octanoyl]-ampicillin

Formula I with R¹, R⁵=H; R², R³=COOCH₃, R⁴=5-Cl, R⁶=R⁹ with R⁸=OCOOCH₃, R⁷=CH₃, n=1, m=1, X and Z=direct bond, Y=ampicillino.

The title compound (7) and the corresponding sodium salt were obtained analogous to example 1 from 3,7-bis-(5-chlor-2,3-dimethoxycarbony-oxybenzoyl)-3,7-diaza-octanoic acid and ampicillin as a colourless solid, yield 40%.

¹HNMR (DMSO-d₆): 1.39 (s, 3H, CH₃); 1.53 (s, 3H, CH₃); 1.75 (m, 2H, CH₂); 2.78 (3H, S, CH₃); 2.96-3.40 (4H, m, 2×CH₂); 3.81 (6H, m, 2×OCH₃); 3.85 (m, 6H, 2×OCH₃); 3.93 (m, 2H, CH₂COOH); 4.18 (s, 1H, 3-CH); 5.38 (d, 1H, 7-CH); 5.48 (q, 1H, 6-CH); 5.74 (d, 1H, α-CH); 7.75-7.20 (m, 9H, aromat.); 8.75 (m, 1H, NHCO); 9.19 (d, 1H, NHCO).

### Example 8

N-{3,7-Bis-[5-brom-2,3-di-(methoxycarbonyloxy)-benzoyl]-3,7-diaza-octanoyl}-ampicillin

Formula I with R¹, R⁵=H; R², R³=COOCH₃, R⁴=5-Br, R⁶=R⁹ with R⁸=COCH₃, R⁷=CH₃, n=1, m=1, X and Z=direct bond, Y=ampicillino.

The title compound (6) and the corresponding sodium salt were obtained analogous to example 1 from 3,7-bis-(5-brom-2,3-dimethoxycarbonyloxy-benzoyl)-3,7-diaza-octanoic acid and ampicillin as a colourless solid, yield 50%.

¹HNMR (DMSO-d₆): 1.75 (2H, m, CH₂); 1.53 (s, 3H, CH₃); 1.39 (s, 3H, CH₃), 2.78 (3H, s, CH₃); 3.85 (6H, m, 2×OCH₃); 3.95 (2H, m, CH₂COOH), 4.18 (s, 1H, 3-CH); 2.96-3.40 (4H, m, 2×CH₂); 3.80 (6H, m, 2×OCH₃); 5.52 (q, 1H, 6-CH); 5.38 (d, 1H, 7-CH); 5.74 (d, 1H, α-CH); 7.85-7.25 (9H, m, aromat.); 8.75 (m, 1H, NHCO); 9.20 (d, 1H, NHCO).

### Example 9

N-[3,7-Bis-(2,3-diacetoxybenzoyl)-3,7-diaza-octanoyl]-ampicillin

Formula I with R¹, R⁴, R⁵=H; R², R³=COCH₃, R⁶=R⁹ with R⁸=COCH₃, R⁷=CH₃, n=1, m=1, X and Z=direct bond, Y=ampicillino.

The title compound (9) and the corresponding sodium salt were obtained analogous to example 1 from 3,7-bis-(2,3-diacetoxybenzoyl)-3,7-diaza-octanoic acid and ampicillin as a colourless solid, yield 40%.

¹HNMR (DMSO-d₆): 1.40 (s, 3H, CH₃); 1.53 (s, 3H, CH₃); 1.75 (2H, m, CH₂); 2.20 (s, 6H, COCH₃); 2.27 (6H, s, COCH₃); 2.75 (s, 3H, CH₃); 2.87-3.15 (m, 2H, CH₂); 3.98 (m, 2H, CH₂COOH), 5.39 (m, 1H, 7-CH); 5.52 (m, 1H, 6-CH); 5.85 (m, 1H, α-CH); 6.94-7.48 (m, 11H, aromat.); 8.73 (m, 1H, NHCO); 9.15 (m, 1H, NHCO).

### Example 10

N-[3,8-Bis-(2,3-diacetoxybenzoyl)-3,8-diaza-octanoyl]-ampicillin

Formula I with R¹, R⁴, R⁵, R⁷=H; R², R³, R⁸=COCH₃, R⁶=R⁹ with R⁸=COCH₃, n=1, m=2, X and Z=direct bond, Y=ampicillino.

The title compound (10) and the corresponding sodium salt were obtained analogous to example 1 from 3,8-bis-(2,3-diacetoxybenzoyl)-3,8-diaza-octanoic acid and ampicillin as a colourless solid, yield 50%.

¹HNMR (DMSO-d₆): 1.40-1.60 (4H, m, CH₂); 1.40 (s, 3H, CH₃); 1.54 (s, 3H, CH₃); 2.17 (3H, s, COCH₃); 2.21 (3H, s, COCH₃); 2.27 (6H, s, COCH₃); 3.30 (3H, s, CH₃); 3.22 (2H, m, CH₂); 3.95 (2H, m, CH₂COOH); 4.19 (s, 1H, 3-CH); 5.38 (m, 1H, 7-CH); 5.51 (m, 1H, 6-CH); 5.72 (m, 1H, α-CH); 6.94-7.52 (11H, m, aromat.); 8.30 (1H, m, NHCO); 8.70 (m, 1H, NHCO); 8.70 (m, 1H, NHCO).

### Example 11

N-[3,8-Bis-(2,3-diacetoxybenzoyl)-3,8-diaza-octanoyl]-amoxicillin

Formula I with R¹, R⁴, R⁵, R⁷=H; R², R³, R⁸=OCOCH₃, R⁶=R⁹ with R⁸=COCH₃, n=1, m=2, X and Z=direct bond, Y=amoxicillino.

The title compound (11) was obtained analogous to example 1 from 3,8-Bis-(2,3-diacetoxybenzoyl)-3,8-diaza-octanoic acid and amoxycillin as a colourless solid, yield 40

¹HNMR (DMSO-d₆): 1.40-1.60 (m, 4H, CH₂); 1.41 (s, 3H, CH₃); 1.54 (s, 3H, CH₃); 2.17 (s, 3H, COCH₃); 2.21 (s, 3H, COCH₃); 2.27 (s, 6H, COCH₃); 3.13 (m, 3H, CH₃); 3.03 (m, 2H, CH₂); 3.95 (m, 2H, CH₂COOH); 4.18 (s, 1H, 3-CH); 5.38 (m, 1H, 7-CH); 5.55 (m, 2H, α-CH+6-CH); 6.60-7.45 (m, 10H, aromat.); 8.32 (m, 1H, NHCO); 8.56 (m, 1H, NHCO); 9.02 (m, 1H, NHCO); 9.38 (s, 1H, OH).

The preparation of the sodium salt was performed by addition of a solution of 0.02 g sodium ethylhexanoate in 3 ml ethyl acetate to a solution of 0.10 g of the title compound in 12 ml tetrahydrofuran. The obtained precipitate was filtered after 10 min standing and than washed with ethyl acetate. The sodium salt of the title compound was obtained as a colourless solid, yield 90%.

### Example 12

N-{3,7-Bis-(2,3-dichlor-5,6-di-methoxycarbonyloxy-benzoyl)-3,7-diaza-octanoyl}-ampicillin

Formula I with R¹, R⁵=H; R², R³=COOCH₃, R⁴=5,6-Di-Cl, R⁶=R⁹, R⁷=CH₃, n=1, m=1, X and Z=direct bond, Y=ampicillino.

The title compound (12) and the corresponding sodium salt were obtained analogous to example 1 from 3,7-bis-(2,3-dichlor-5,6-di-methoxycarbonyloxy-benzoyl)-3,7-diaza-octanoic acid and ampicillin as a colourless solid, yield 30%.

¹HNMR (DMSO-6): 1.39 (s, 3H, CH₃); 1.53 (s, 3H, CH₃); 1.80 (m, 2H, CH₂); 2.78 (s, 3H, CH₃); 2.96-3.40 (m, 4H, 2×CH₂); 3.82 (m, 6H, 2×OCH₃); 3.85 (m, 6H, 2×OCH₃); 4.05 (m, 2H, CH₂COOH); 4.19 (s, 1H, 3-CH); 5.38 (m, 1H, 7-CH); 5.48 (m, 1H, 6-CH); 5.70 (m, 1H, α-CH); 7.20-8.05 (m, 7H, aromat.); 8.65 (m, 1H, NHCO); 9.19 (d, 1H, NHCO).

### Example 13

N-{[6-Bis-2,3-(diacetoxybenzoyl)-2-aminoethyl]-3-(2,3-diacetoxybenzoyl)-3,6-diaza-hexanoyl}-ampicillin

Formula I with R¹, R⁴, R⁵=H, R², R³=COCH₃, R⁶ and R⁷=R¹³ with p=2, n=0, m=1, X and Z=direct bond, Y=ampicillino.

The title compound (13) and the corresponding sodium salt were obtained analogous to example 1 from 6-[bis-(2,3-diacetoxybenzoyl)-2-aminoethyl]-3-(2,3-diacetoxy-benzoyl)-3,6-diazahexanoic acid and ampicillin as a colourless solid, yield 60%.

¹H NMR (DMSO-d₆): 1.40 (s, 3H, CH₃); 1.54 (s, 3H, CH₃); 2.19-2.28 (m, 18H, COCH₃); 3.12-4.07 (m, 14H, NCH₂); 4.20 (s, 1H, 3-CH); 5.38-5.39 (m, 1H, 7-CH); 5.50-5.51 (m, 1H, α-CH); 5.72-5.75 (m, 1H, 6-CH); 7.07-7.81 (m, 14H, aromat.); 8.54-9.20 (m, 2×1H, NHCO).

### Example 14

N-[3,7-Bis-(2,3-dimethoxycarbonyloxybenzoyl)-3,7-diaza-5-hydroxy-heptanoyl]-ampicillin

Formula I with R¹, R⁴=H, R⁵=OH, R², R³=COOCH₃, R⁶=R⁹ with R⁸=COOCH₃, R⁷=H, n=1, m=1, X and Z=direct bond, Y=ampicillino.

The title compound (16) and the corresponding sodium salt were obtained analogous to example 1 from 3,7-bis-(2,3-dimethoxycarbonyloxybenzoyl)-3,7-diaza-5-hydroxy-heptanoic acid and ampicillin as a colourless solid, yield 65%.

¹HNMR (DMSO-d₆): 1.40 (s,3H,CH₃); 1.55 (s,3H,CH₃); 2.80-3.10 (m, 4H, 2×CH₂); 3.75 (s,3H,CH₃); 3.84 (s, 6H, OCH₃); 3.90 (s, 3H, OCH₃); 4.15 (s, 2H, CH₂COOH); 4.19 (s, 1H, 3-CH); 5.38 (m, 1H, 7-CH); 5.50 (m, 1H, α-CH); 5.55 (q, 1H, 6-CH); 7.2-7.95 (m,11H, aromat.); 8.97 (d,1H, NHCO); 9.12 (d,1H, NHCO).

### Example 15

N-{4-[5-(Bis-N-2,3-diacetoxybenzoyl-2-aminoethyl)-2-(2,3-di-acetoxybenzoyl)-2,5-diaza-pentyl]-benzoyl-ampicillin

Formula I with R¹, R⁴, R⁵=H, R², R³=COCH₃, R⁶ and R⁷=R¹³ with p=2, n=0, m=1, X=direct bond, Y=ampicillino, Z=p-phenylene.

The title compound (18) and the corresponding sodium salt were obtained analogous to example 1 from 4-[5-(bis-N-2,3-diacetoxybenzoyl-2-aminoethyl)-2-(2,3-diacetoxy-benzoyl)-2,5-diaza-pentyl]-benzoic acid and ampicillin as a colourless solid, yield 47%.

The product was obtained by acidification of the solution of the reaction as solid precipitate, which was separated from the solvent, washed with water and dried under vaccuum.

¹H NMR (DMSO-d₆): 1.40 (s, 3H, CH₃); 1.52 (s, 3H, CH₃); 2.15-2.25 (m, 18H, COCH₃); 2.66-3.56 (m, 14H, NCH₂); 4.18 (s, 1H, 3-CH); 5.37-5.41 (m, 1H, 7-CH); 5.50-5.56 (m, 1H, α-CH); 5.90 (m, 1H, 6-CH); 7.27-8.25 (m, 18H, aromat.); 8.79-9.03 (m, 2×1H, NHCO).

### Example 16

N-{4-[2,6-bis-(2,3-di-methoxycarbonyloxybenzoyl)-2,6-diaza-heptyl]-benzoyl}-ampicillin

Formula I with R¹, R⁴, R⁵=H, R², R³, R⁸=COOCH₃, R⁶=R⁹, R⁷=CH₃, n=1, m=1, X=direct bond, Y=ampicillino, Z=p-phenylene.

The title compound (20) and the corresponding sodium salt were obtained analogous to example 1 from 4-[2,6-bis-(2,3-di-methoxycarbonyloxybenzoyl)-2,6-diaza-heptyl]-benzoic acid and ampicillin as a colourless solid, yield 60%.

¹HNMR (DMSO-d₆): 1.39; 1.46 (2×s, 6H, CH₃,); 1.65-1.85 (m, 4H, CH₂); 2.73-2.75 (m, 3H, NCH₃); 3.73-3.86 (m, 12H, COOCH₃); 4.17 (s, 1H, CH); 5.51-5.87 (m, 3H, CH); 7.28-7.91 (m, 15H, aromat.), 9.07-9.09 (m, 2H, NHCO).

### Example 17

N-{4-[2,7-Bis-(2,3-diacetoxybenzoyl)-2,7-diaza-heptyl]-phenoxyacetyl}-ampicillin

Formula I with R¹, R⁴, R⁵=H, R², R³, R⁸=COCH₃, R⁶=R⁹, n=2, m=1, X=direct bond, Y=ampicillino, Z=p-C₆H₄—O—CH₂—.

The title compound (21) and the corresponding sodium salt were obtained analogous to example 1 from 4-[2,7-bis-(2,3-diacetoxybenzoyl)-2,7-diaza-heptyl]-phenoxy-acetic acid and ampicillin as a colourless solid, yield 65%.

¹H NMR (DMSO-d₆): 1.39-1.53 (m, 10H, 2×CH₃, 2×CCH₂); 2.16-2.27 (m, 12H, 4×COCH₃); 2.90-3.15 (4 m, H, 2×NCH₂); 4.18 (s, 2H, OCH₂CO); 4.63 (s, 2H, CONCH₂Ar); 5.39 (d, J=4.04.1H, CH); 5.51-5.52 (m, 1H, CH); 5.75-5.85 (m, 1H, CH); 6.88-7.41 (m, 15H, aromat.); 8.20-8.35 (m, 1H, NHCO); 8.54-8.57 (m, 1H, NHCO); 9.18-9.20 (m, 1H, NHCO).

### Example 18

N-{2-[2,7-Bis-(2,3-diacetoxybenzoyl)-2,7-diaza-heptyl]-benzooyl}-ampicillin

Formula I with R¹, R⁴, R⁵, R⁷=H, R², R³, R⁸=COCH₃, R⁶=R⁹, R¹⁴=H, with n=2, m=1, X=direct bond, Y=ampicillino, Z=o-phenylene.

The title compound (22) and the corresponding sodium salt were obtained analogous to example 1 from 2-[2,7-bis-(2,3-diacetoxybenzoyl)-2,7-diaza-heptyl]-benzoic acid and ampicillin as a colourless solid, yield 36%.

¹H NMR (DMSO-d₆): 1.16-1.45 (m, 4H, CH₂CH₂); 1.40 (s, 3H, CH₃); 1.52 (s, 3H, CH₃); 2.15-2.26 (m, 12H, COCH₃); 2.95-3.23 (m, 6H, NCH₂); 4.19 (s, 1H, 3-CH); 5.39-5.40 (m, 1H, 7-CH); 5.48-5.59 (m, 1H, α-CH); 5.88-5.91 (m, 1H, 6-CH); 7.24-7.54 (m, 15H, aromat.); 8.16-8.32 (m, 1H, NHCO); 8.92-9.12 (m, 1H, NHCO).

### Example 19

N-{2-[2,6-Bis-(2,3-diacetoxybenzoyl)-2,6-diaza-heptyl]-benzoyl}-ampicillin

Formula I with R¹, R⁴, R⁵=H, R⁷=CH₃, R², R³, R⁸=COCH₃, R⁶=R⁹, R¹⁴=H, with n=1, m=1, X=direct bond, Y=ampicillino, Z=o-phenylene.

The title compound (23) and the corresponding sodium salt were obtained analogous to example 1 from 2-[2,6-bis-(2,3-diacetoxybenzoyl)-2,6-diaza-heptyl]-benzoic acid and ampicillin as a colourless solid, yield 20%.

¹H NMR (DMSO-d₆): 1.45-1.82 (m, 2H, CH₂CH₂); 1.40 (s, 3H, CH₃); 1.52 (s, 3H, CH₃); 2.12-2.27 (m, 12H, COCH₃); 2.71 (s, 3H, CH₃); 2.74-3.29 (m, 6H, NCH₂); 4.18 (s, 1H, 3-CH); 5.38-5.40 (m, 1H, 7-CH); 5.50-5.55 (m, 1H, α-CH); 5.84-5.93 (m, 1H, 6-CH); 6.88-7.49 (m, 15H, aromat.); 8.96-9.26 (m, 2H, NHCO).

### Example 20

N-{4-[2,6,9,13-Tetrakis-(2,3-diacetoxybenzoyl)-2,6,9,13-tetraaza-tridecyl]-benzoyl}-ampicillin

Formula I with R¹, R⁴, R⁵=H, R², R³, R⁸=COCH₃, R⁶=R⁹, R⁷=R¹⁴ with s=2 and R¹³ with p=3; n=1, m=1, X=direct bond, Z=p-phenylene, Y=ampicillino

The title compound (24) and the corresponding sodium salt were obtained analogous to example 1 from 4-[2,6,9,13-tetrakis-(2,3-diacetoxy-benzoyl)-2,6,9,13-tetraaza-tridecyl]-benzoic acid and ampicillin as a colourless solid, yield 50%.

¹H NMR (DMSO-d₆): 1.38; 1.51 (s, 6H, 2×CH₃); 1.40-1.70 (m, 4H, CCH₂); 2.06-2.27 (m, 24H, COCH₃); 3.03-3.30 (m, 12H, NCH₂); 4.17 (s, 1H, CH); 4.37-4.39 (m, 1H, CH); 5.51-5.54 (m, 1H, CH); 5.88-5.94 (m, 1H, CH); 7.19-7.51 (m, 21H, aromat.); 8.25-9.03 (m, 3H, NHCO).

### Example 21

N-{6-[2,7-Bis-(2,3-Diacetoxybenzoyl)-2,7-diaza-heptyl]-2,3-dimethoxy-benzoyl}-ampicillin

Formula I with R¹, R⁴, R⁵, R⁷=H, R², R³, R⁸=COCH₃, R⁶=R⁹, with n=2, m=1, X=direct bond, Y=ampicillino, Z=o-phenylene with R¹⁵=3,4-dimethoxy.

The title compound (25) and the corresponding sodium salt were obtained analogous to example 1 from 6-[2,7-bis-(2,3-diacetoxybenzoyl)-2,7-diaza-heptyl]-2,3-dimethoxy-benzoic acid and ampicillin as a colourless solid, yield 11%.

¹H NMR (DMSO-d₆): 1.03-1.90 (m, 4H, CH₂CH₂); 1.40 (s, 3H, CH₃); 1.53 (s, 3H, CH₃); 2.15-2.26 (m, 12H, COCH₃); 2.80-3.69 (m, 6H, NCH₂); 3.28 (s, 6H, OCH₃), 4.19 (s, 1H, 3-CH); 5.37-5.40 (m, 1H, 7-CH); 5.42-5.55 (m, 1H, α-CH); 5.78-5.92 (m, 1H, 6-CH); 6.94-7.73 (m, 13H, aromat.); 8.18-8.29 (m, 1H, NHCO); 8.93-9.11 (m, 2H, 2×NHCO).

### Example 22

N-{2-[2,6-Bis-(2,3-di-methoxycarbonyloxy-benzoyl)-2,6-diaza-heptyl]-benzoyl}-ampicillin

Formula I with R¹, R⁴, R⁵=H, R⁷=CH₃, R², R³, R⁸=COOCH₃, R⁶=R⁹, R¹⁵=H, with n=1, m=1, X=direct bond, Y=ampicillino, Z=o-phenylene.

The title compound (26) and the corresponding sodium salt were obtained analogous to example 1 from 2-[2,6-bis-(2,3-di-methoxycarbonyloxy-benzoyl)-2,6-diaza-heptyl]-benzoic acid and ampicillin as a colourless solid, yield 21%.

¹H NMR (DMSO-d₆): 1.32-1.68 (m, 2H, CH₂); 1.40 (s, 3H, CH₃); 1.52 (s, 3H, CH₃); 2.73 (s, 3H, CH₃); 2.68-3.04 (m, 4H, NCH₂); 3.74-3.84 (m, 12H, COOCH₃); 4.18 (s, 1H, 3-CH); 4.39-4.49 (m, 2H, NCH₂); 5.39-5.40 (m, 1H, 7-CH); 5.43-5.53 (m, 1H, α-CH); 5.81-5.89 (m, 1H, 6-CH); 6.78-7.50 (m, 15H, aromat.), 9.03-9.13 (m, 1H, NHCO).

### Example 23

N-{2-[2,6,10-Tris-(2,3-diacetoxybenzoyl)-2,6,10-triaza-decyl]-benzoyl}-ampicillin

Formula I with R¹, R⁴, R⁵, R⁷=H, R², R³, R⁸=COCH₃, R⁶=R⁹, R¹⁵=H, with n=1, m=2, X=direct bond, Y=ampicillino, Z=o-phenylene, C₆₃H₆₄N₆O₂₀S (1257)

The title compound (27) and the corresponding sodium salt were obtained analogous to example 1 from 2-[2,6,10-tris-(2,3-diacetoxybenzoyl)-2,6,10-triaza-decyl]-benzoic acid and ampicillin as a colourless solid, yield 17%.

¹H NMR (DMSO-d₆): 1.62-1.73 (m, 4H, CH₂); 1.40 (s, 3H, CH₃); 1.51 (s, 3H, CH₃); 2.08-2.27 (m, 18H, COCH₃); 2.50-3.56 (m, 10H, NCH₂); 4.18 (s, 1H, 3-CH); 5.39-5.40 (m, 1H, 7-CH); 5.51-5.52 (m, 1H, α-CH); 5.85-5.90 (m, 1H, 6-CH); 6.80-7.54 (m, 18H, aromat.), 8.10-8.32 (m, 1H, NHCO); 8.88-9.07 (m, 2H, 2×NHCO).

### Example 24

N-[3,7-Bis-(2,3-diacetoxybenzoyl)-3,7-diaza-heptanoyl]-ampicillin

Formula I with R¹, R⁴, R⁵, R⁷=H, R², R³, R⁸=COCH₃, R⁶=R⁹, n=1, m=1, X and Z=direct bond, Y=ampicillino.

The title compound (28) and the corresponding sodium salt were obtained analogous to example 1 from 3,7-bis-(2,3-diacetoxybenzoyl)-3,7-diaza-heptanoic acid and ampicillin as a colourless solid, yield 30%.

¹HNMR (DMSO-d₆): 1.40-1.60 (m, 8H, CCH₂, CH₃); 2.14-2.23 (m, 12H, COCH₃); 3.22 (m, 4H, NCH₂), 3.95 (m, 2H, NCH₂CO), 4.14 (s, 1H, 3-CH), 5.33 (m, 1H, 7-CH); 5.47 (m, 1H, 6-CH); 5.85 (m, 1H, α-CH); 7.03-7.52 (m, 11H, aromat.); 8.25 (m, 1H, NHCO); 8.68 (m, 1H, NHCO); 9.11 (m, 1H, NHCO).

### Example 25

N-[3,9-Bis-(2,3-diacetoxybenzoyl)-3,9-diaza-nonanoyl]-ampicillin

Formula I with R¹, R⁴, R⁵, R⁷=H, R², R³, R⁸=COCH₃, R⁶=R⁹, n=3, m=1, X and Z=direct bond, Y=ampicillino.

The title compound (29) and the corresponding sodium salt were obtained analogous to example 1 from 3,9-bis-(2,3-diacetoxybenzoyl)-3,9-diaza-nonanoic acid and ampicillin as a colourless solid 40%.

¹HNMR (DMSO-d₆): 1.40-1.540 (m, 12H, CCH₂, CH₃); 2.16-2.23 (m, 12H, COCH₃); 3.16-3.31 (m, 4H, NCH₂); 4.15 (m, 2H, CH₂CO); 3.90 (m, 1H, CH); 5.35 (m, 1H, CH); 5.48 (m, 1H, CH); 5.72 (m, 1H, CH); 7.25-7.49 (m, 11H, aromat.); 8.32 (m, 1H, NHCO); 8.68 (m, 1H, NHCO); 9.11-9.15 (m, 1H, NHCO).

### Example 26

N-[3,6-Bis-(2,3-diacetoxybenzoyl)-3,6-diaza-hexanoyl]-ampicillin

Formula I with R¹, R⁴, R⁵, R⁷=H, R², R³, R⁸=COCH₃, R⁶=R⁹, n=0, m=1, X and Z=direct bond, Y=ampicillino.

The title compound (30) and the corresponding sodium salt were obtained analogous to example 1 from 3,6-bis-(2,3-diacetoxybenzoyl)-3,6-diaza-hexanoic acid and ampicillin as a colourless solid, yield 23%.

¹HNMR (DMSO-d₆): 1.40; 1.52 (m, 6H, CH₃); 2.05-2.27 (m, 12H, COCH₃); 3.10-3.40 (m, 6H, NCH₂); 4.14 (s, 1H, CH); 5.35-5.38 (m, 1H, CH); 5.49-5.51 (m, 1H, CH); 5.72 (m, 1H, CH); 7.24-7.49 (m, 11H, aromat.); 8.25-8.35 (m, 1H, NHCO); 8.72-8.74 (m, 1H, NHCO); 9.10-9.20 (m, 1H, NHCO).

TABLE
Antibacterial activity of the siderophore-antibiotic conjugates MICs [ug/ml]

| | Pseudomonas | | | | | | |
|---|---|---|---|---|---|---|---|
| | aeruginosa | | E. coli | Klebsiella | Stenotroph. | Serratia | Staph. |
| | | ATCC | ATCC | pneumoniae | maltoph. | marc. | aureus |
| Example | SG137 | 27853 | 25922 | ATCC10031 | GN12873 | SG621 | SG511 |
| 1 | 0.031 | 0.031 | 0.062 | 0.008 | 0.062 | 0.078 | 10 |
| 2 | <0.05 | 3.125 | 0.4 | 0.4 | n.d. | n.d. | 6.25 |
| 3 | 0.1 | 0.78 | 0.2 | <0.05 | 0.4 | 0.2 | 12.5 |
| 4 | 0.2 | 1.56 | 0.2 | <0.05 | 50 | n.d. | 6.25 |
| 5 | 25 | 25 | 100 | 0.2 | 50 | 0.4 | n.d. |
| 6 | 0.2 | 1.56 | 1.56 | 0.1 | 0.4 | 0.78 | 6.25 |
| 7 | <0.005 | 0.1 | 0.05 | <0.005 | 0.02 | 0.02 | 12.5 |
| 8 | <0.005 | 0.05 | <0.005 | <0.005 | 0.02 | 0.02 | 6.25 |
| 9 | 0.005 | 0.08 | 0.02 | <0.005 | 0.02 | 0.05 | 3.12 |
| 10 | <0.005 | 0.05 | <0.005 | <0.005 | <0.005 | <0.005 | 3.12 |
| 11 | 0.01 | 0.1 | 0.01 | <0.005 | <0.005 | <0.005 | 3.12 |
| 12 | 0.05 | 0.1 | 0.02 | <0.005 | 0.05 | 0.05 | 3.12 |
| 13 | 0.1 | 3.12 | 0.78 | 0.1 | 0.2 | 0.78 | 1.56 |
| 14 | 0.1 | 1.56 | 6.25 | 0.2 | 0.4 | 1.56 | 0.4 |
| 15 | <0.05 | 0.2 | 0.78 | 0.1 | <0.05 | 0.4 | 0.78 |
| 16 | <0.05 | 0.78 | 0.4 | 0.1 | <0.05 | 1.56 | 1.56 |
| 17 | 0.2 | 6.25 | 0.78 | <0.05 | 0.2 | 6.25 | 1.56 |
| 18 | 0.04 | 0.2 | 0.4 | <0.05 | <0.05 | 0.24 | 6.25 |
| 19 | 0.1 | 3.12 | 0.78 | 0.1 | 0.2 | 0.78 | 1.56 |
| 20 | 0.4 | 1.56 | 1.56 | 0.78 | 0.2 | 1.56 | 6.25 |
| 21 | 0.78 | 12.5 | 3.12 | 0.4 | 0.2 | 1.56 | 0.2 |
| 22 | 0.2 | 0.4 | 1.56 | 0.4 | <0.05 | 0.78 | 6.25 |
| 23 | 0.78 | 1.56 | 6.25 | 0.78 | 0.2 | 3.12 | 12.5 |
| 24 | 3.12 | 3.12 | 3.12 | 0.2 | 0.2 | 0.78 | 3.12 |
| 25 | 0.04 | 0.16 | 0.16 | <0.005 | 0.02 | 0.04 | 2.5 |
| 26 | 0.4 | 0.78 | 0.78 | 0.1 | 0.78 | 0.78 | 3.12 |
| azlocillin | 6.25 | 6.25 | 6.25 | 6.25 | 25 | 50 | 0.4 |
| ampicillin | >100 | >100 | 6.25 | 6.25 | >100 | 25 | 0.4 |
| meropenem | 0.2 | 0.4 | 0.04 | 0.04 | >100 | 0.06 | 0.1 |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A Catecholate-β-lactam conjugate corresponding to formula I, 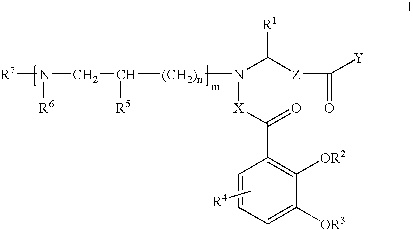
wherein
R¹ is selected from the group consisting of H, alkyl, substituted alkyl, aryl, and substituted aryl;
R² is H, —CO-alkyl, or —COO-alkyl,
X is a direct bond between the nitrogen atom and carbon atom connected to X in formula I,
or X is (CH₂)_{q}NH— or CO(CH₂)_{q}NH— with q=1-6,
or R² and X together are 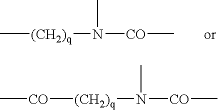
with q=1-6;
R³ is H, —CO-alkyl, or —COO-alkyl;
R⁴ is present at each available substitution location, and each R⁴ is independently selected from the group consisting of H, alkyl, substituted alkyl, aryl, substituted aryl, halogen, alkoxy, and substituted alkoxy;
R⁵ is H, OH, —O-alkyl, —O-acyl, —O-aryl, alkyl, substituted alkyl, aryl, or substituted aryl;
R⁶=R⁹, which is 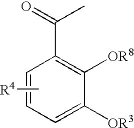
where R⁸ is H, COalkyl, COalkyl,
or
R⁶=R¹⁰ which is 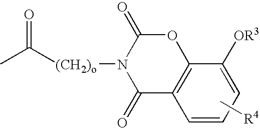
mit o=1
where o=1-10
or
R⁶=R¹¹, which is 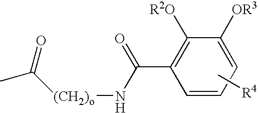
where o=1-10
or
R⁶=R¹³ 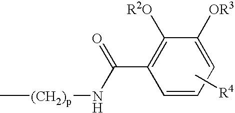
where p=2-10,
R⁷=H, alkyl, substituted alkyl, aryl, substituted aryl, or R¹³,
or
R⁷=R¹⁴, which is 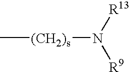
where s=2-4,
or R⁶ and R⁷ are each R¹², which is 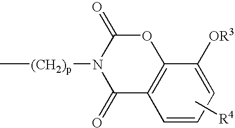
where p=2-10;
n=0-8;
m=1-3;
Y is a residue of a β-lactam antibiotic; and
Z is a direct bond between the carbon atoms connected to Z in formula I, or
Z is —(CH₂)ᵣ— with r=0-10, or
Z is arylene or substituted arylene;
or a salt thereof, or an ester thereof that is easily cleaved under physiological conditions.

2. A compound according to claim 1, wherein the compound contains at least 1 asymmetric carbon atom, and wherein the compound is present as a corresponding D- or L-form, in the form of one or more diastereomers, in the form of one or more enantiomers, as a racemic mixture, or as a mixture of diastereomers and enantiomers.

3. A compound according to claim 1, wherein Y is a penicillin derivative.

4. A compound according to claim 1, wherein Y is an ampicillin residue according to formula A where R is H, a amoxicillin residue according to formula A where R is OH, a bacampicillin residue, a cephalosporin residue, or a cefaclor residue according to formula B.

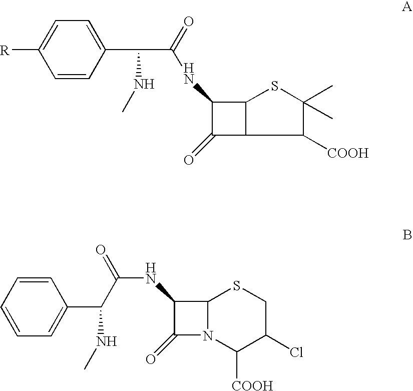

5. A compound according to claim 1, wherein Z is 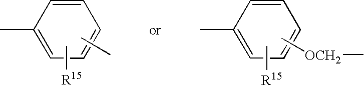
wherein R¹⁵ is present at each available substitution location, and wherein each R¹⁵ is independently selected from the group consisting of H, alkyl, substituted alkyl, aryl, substituted aryl, halogen, alkoxy, and substituted alkoxy,

6. A compound according to claim 1, wherein
R¹ and R⁵ are H;
R² is H, —CO-alkyl, or —COO-alkyl;
R³ is H, —CO-alkyl, or —COO-alkyl;
R⁴ is H or halogen;
R⁶ is R⁹ or R¹³, R⁷ is H, CH₃, or R¹³, or R⁶ and R⁷ are each R¹², where p=2-10;
n=1-2;
m=1-2;
X and Z are direct bonds;
and Y is a residue of ampicillin or amoxicillin.

7. A compound according to claim 1, wherein
R¹ and R⁵ are H;
R² is H, —CO-alkyl, or —COO-alkyl;
R³ is H, —CO-alkyl, or —COO-alkyl;
R⁴ is H or halogen;
R⁶ is R⁹ or R¹³, R⁷ is H, CH₃, or R¹³, or R⁶ and R⁷ are each R¹², where p=2-10;
n=1-3;
m=1;
X is a direct bond;
Z=phenylene or substituted phenylene; and
Y is a residue of ampicillin or amoxicillin.

8. A compound according to claim 1, wherein
R¹ and R⁵ are H;
R² is H, —CO-alkyl, or —COO-alkyl;
R³ is H, —CO-alkyl, or —COO-alkyl;
R⁴ is H or halogen;
R⁶ is R⁹;
R⁷ is H or CH₃;
n=1-3;
m=1;
X and Z are direct bonds; and
Y is a residue of ampicillin or amoxicillin.

9. A compound according to claim 1, wherein
R¹ and R⁵ are H;
R² is H, —CO-alkyl, or —COO-alkyl;
R³ is H, —CO-alkyl, or —COO-alkyl;
R⁴ is H or halogen;
R⁶ and R⁷ are R¹³ where p=2;
n=0;
m=1;
X and Z are direct bonds; and
Y is a residue of ampicillin or amoxicillin.

10. A compound according to claim 1, wherein
R¹ is alkyl;
R² is H, —CO-alkyl, or —COO-alkyl;
R³ is H, —CO-alkyl, or —COO-alkyl;
R⁴ is H or halogen;
R⁵ is H;
R⁷ is H or CH₃;
R⁶ is R⁹;
n=1-3;
m=1;
X and Z are direct bonds; and
Y is a residue of ampicillin or amoxicillin.

11. A compound according to claim 1, wherein
R¹ and R⁵ are H;
R³ is H, —CO-alkyl, or —COO-alkyl;
R⁴ is H or halogen;
R⁷ is H or CH₃;
R is R¹⁰ with o=1-2;
n=1-3;
m=1-2;
X together with R² is 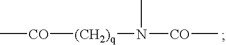
Z is a direct bond; and
Y is a residue of ampicillin or amoxicillin.

12. A method for treating a bacterial infection comprising administering an effective amount of a compound according to claim 1, or a pharmaceutically acceptable salt thereof, or a pharmaceutically acceptable ester thereof that can be cleaved under physiological conditions.

13. A pharmaceutical composition comprising a compound according to claim 1, or a pharmaceutically acceptable salt thereof, or a pharmaceutically acceptable ester thereof that can be cleaved under physiological conditions, and a pharmaceutically acceptable carrier or adjuvant.
